(19) United States
(12) Patent Application Publication
(10) Pub. No.: US 2006/0104907 A1
(43) Pub. Date: May 18, 2006

(54) Biologically potent analogues of the Dmt-Tic pharmacophore and methods of use

(75) Inventors: Lawrence H. Lazarus, Durham, NC (US); Severo Salvadori, Ferrara (IT); Gianfranco Balboni, Ferrara (IT); Remo Guerrini, Ferrara (IT)

Correspondence Address: LEYDIG, VOIT & MAYER, LTD., TWO PRUDENTIAL PLAZA, SUITE 4900, 180 NORTH STETSON AVENUE, CHICAGO, IL, 60601-6780, US

(73) Assignee: Government of the U.S.A., represented by the Secretary, Department of Health and Human Services, Rockville, MD (US)

(21) Appl. No.: 11/280,752

(22) Filed: Nov. 16, 2005

Related U.S. Application Data
(60) Provisional application No. 60/628,147, filed on Nov. 16, 2004.

(51) Int. Cl. A61K 49/00; G01N 33/53; C07K 5/06; C07D 405/02; C07D 217/12

(52) U.S. Cl. 424/9.6; 530/331; 435/7.1; 546/146

(57) ABSTRACT

The present invention provides a compound of formula: 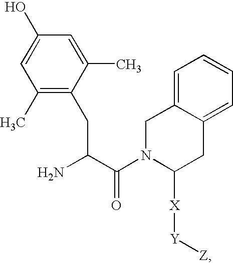 wherein X is a group comprising one or more amino acid residues, Y is a spacer, and Z comprises a fluorescent molecule, and compositions and methods of identifying δ- and μ-opioid receptors.

### CROSS-REFERENCE TO RELATED APPLICATIONS

This patent application claims the benefit of U.S. Provisional Patent Application No. 60/628,147, filed Nov. 16, 2004.

### FIELD OF THE INVENTION

This invention pertains to a fluorescent peptide-based probe comprising the Dmt-Tic (2′,6′-dimethyl-L-tyrosine-1,2,3,4-tetrahydroisoquinoline-3-carboxylic acid) pharmacophore. The present invention also relates to compositions thereof and methods of identifying δ- and μ-opioid receptors.

### BACKGROUND OF THE INVENTION

Endogenous opioids are believed to be involved in the modulation of pain perception, in mood and behavior, learning and memory, diverse neuroendocrine functions, immune regulation and cardiovascular and respiratory function. Opioids also have a wide range of therapeutic utilities, such as treatment of opiate and alcohol abuse, neurological diseases, neuropeptide or neurotransmitter imbalances, neurological and immune system dysfunctions, graft rejections, pain control, shock and brain injuries.

There are believed to be three types of opiate receptors, namely δ, κ and μ. Genes encoding these three main receptor types now have been cloned. Sequencing of the cloned opioid receptor genes has revealed a substantial degree of amino acid homology between different receptor types (Meng et al., PNAS USA, 90: 9954-9958 (1993); Thompson et al., Neuron, 11: 903-913 (1993); Evans et al., Science, 258: 1952-1955 (1992); and Kieffer et al., PNAS USA, 89: 12048-12052 (1992)), which explains the tendency of opioid receptor ligands, even those reported to be selective, to bind to more than one type of opioid receptor. Based on differences in the binding profiles of natural and synthetic ligands, subtypes of opioid receptors have been suggested, including μ1 and μ2 (Pasternak et al., Life Sci,. 38: 1889-1898 (1986)) and κ1 and κ2 (Zukin et al., PNAS US,A 85: 4061-4065 (1988)). Different subtypes of a given type of opioid receptor may co-exist in a single cell (Evans et al. (1992), supra; and Kieffer et al. (1992), supra).

The μ-opioid receptor in the brain appears to mediate analgesia (Kosterlitz et al., Br. J. Pharmacol., 68: 333-342 (1980)). It is also believed to be involved with other undesirable effects, such as respiratory depression (Ward et al., Soc. Neurosci. Symp., 8: 388 (abstract) (1982)), suppression of the immune system (Plotnikoffet al., Enkephalins and Endorphins: Stress and the Immune System, Plenum Press, NY (1986); Yahya et al., Life Sci., 41: 2503-2510 (1987)) and addiction (Roemer et al., Life Sci., 27: 971-978 (1981)). Its side effects in the periphery include inhibition of intestinal motility (Ward et al., Eur. J. Pharmacol., 85: 163-170 (1982)) and secretion in the small intestine (Coupar, Br. J. Pharmacol., 80: 371-376 (1983)).

δ-opioid receptors also mediate analgesia but are not involved in addiction. They may have an indirect role in immune suppression.

There appears to be a single binding site for agonists and antagonists in the ligand-binding domain of δ-receptors. Thus, the “message domain” of δ-agonists and δ-antagonists probably presents a similar low energy conformer in order to fit the receptor cavity. The minimum size of that “message domain” constitutes the dimensions of a dipeptide (Temussi et al., Biochem. Biophys. Res. Commun., 198: 933-939 (1994); Mosberg et al., Lett. Pept. Sci., 1: 69-72 (1994); and Salvadori et al., J. Med. Chem., 42: 3100-3108 (1997)), which has a specific spatial geometry in solution (Bryant et al., Trends Pharmacol. Sci., 18: 42-46 (1998); Bryant et al., Biol. Chem., 378: 107-114 (1997); Crescenzi et al., Eur. J. Biochem., 247: 66-73 (1997); and Guerrini et al., Bioorg. Med Chem., 6: 57-62 (1998)) as seen in the crystallographic evidence for TIPP analogues (Flippen-Anderson et al., J. Pept. Res., 49: 384-393 (1997)) and N,N(Me)₂-Dmt-Tic-OH.

The uniqueness of the δ receptor has led to the use of moderately δ-selective alkaloid antagonists in clinical trials, such as for the amelioration of the effects of alcoholism (Froehlich et al., Alcohol. Clin. Exp. Res., 20: A181-A186 (1996)), the treatment of autism (Lensing et al., Neuropsychobiol., 31: 16-23 (1995)), and Tourette's syndrome (Chappell, Lancet, 343: 556 (1994)). The δ-opiate antagonist naltrindole (Portoghese et al., Eur. J. Pharm., 146: 185-186 (1998)) has been shown to inhibit the reinforcing properties of cocaine (Menkens et al., Eur. J. Pharm., 219: 346-346 (1992)), to moderate the behavioral effects of amphetamines (Jones et al., J. Pharmacol. Exp. Ther., 262: 638-645 (1992)), and to suppress the immune system (Jones et al. (1992), supra) for successful organ transplantation (House et al., Neurosci. Lett., 198: 119-122 (1995)) in animal models (Arakawa et al., Transplant Proc., 24: 696-697 (1992); Arakawa et al., Transplant, 53: 951-953 (1992); and Arakawa et al., Transplant. Proc., 25: 738-740 (1993)). The same effects also have been shown for 7-benzylspiroindanylnaltrexone (Lipper et al., Eur. J. Pharmacol., 354: R3-R5 (1998)).

Among the diverse body of opioid ligands, the prototypic dipeptide Dmt-Tic, which evolved from the weakly active Tyr-Tic as a simplification of the TIP(P) class of compounds, represents the minimal peptide sequence that selectively interacts with δ-opioid receptors with potent antagonist activity (Kᵢ^{μ}/Kᵢ^{δ}=150,780; pA₂=8.2) (Salvadori et al., Mol. Med, 1: 678-689 (1995); Temussi et al., Biochem. Biophys. Res. Commun., 198: 933-939 (1994); and Schiller et al., Proc. Natl Acad Sci. USA, 89: 11871-11875 (1992)). Observations of differences between the δ-opioid receptor binding of Dmt-Tic peptides and their Tyr-Tic cognates (Salvadori et al. (1995), supra; Lazarus et al. (1998), supra; and Lazarus et al., Int'l Symp. on Peptide Chem. and Biol., Changchung, PRC (1999)) indicates that Dmt assumes a predominant role in the alignment or anchoring of the peptide within δ-, μ- and κ-opioid receptor binding sites (Bryant et al. (1998), supra; and Bryant et al. (1997), supra; Crescenzi et al. (1997), supra; and Guerrini et al. (1998), supra) or affects the conformation of the dipeptide antagonists in solution (Bryant et al. (1997), supra; and Crescenzi et al. (1997), supra). Furthermore, observations of differences between the spectra of activity exhibited by the Tyr-Tic cognates of certain Dmt-Tic peptides (Schiller et al., PNAS USA, 89: 11871-11875 (1992); Schiller et al., J. Med Chem., 36: 3182-3187 (1993); Schiller et al., Peptides, Hodges and Smith, eds., ESCOM, 1994; pp. 483-486; Temussi et al. (1994), supra; Mosberg et al. (1994), supra; Salvadori et al. (1995), supra; Lazarus et al. (1998), supra; and Lazarus et al. (1999), supra) and the corresponding Dmt-Tic peptides suggests that the C-terminal “address” portion of the peptide can influence the “message domain.”

Recently, cyclic peptides and di- and tri-peptides comprising the pharmacophore Dmt-Tic have been developed and have been shown to exhibit high selectivity, affinity and potency for the δ-opioid receptor. Such peptides have been shown to function as agonists, partial agonists, antagonists, partial antagonists or mixed antagonists/agonists for opioid receptors (see Lazarus et al., U.S. Pat. No. 5,780,589, and Schiller, U.S. Pat. No. 5,811,400).

A variety of modifications to the Tic residue differentially changes receptor selectivity (Santagada et al., Med. Chem. Lett., 10: 2745-2748 (2000); Page et al., Bioorg. Med. Chem. Lett., 10: 167-170 (2000); Salvadori et al., Mol. Med, 1: 678-689 (1995); Balboni et al., Peptides, 21: 1663-1671 (2000); and Capasso et al., FEBS Lett., 417: 141-144 (1997)).

The availability of highly selective ligands for individual receptor types aid in the development of potential therapeutic agents. Moreover, such ligands, acting as either agonists or antagonists, are valuable pharmacological tools to understand the pharmacophoric requirements for binding and the various biological effects produced by individual receptor interactions (Aldrich, J. V. Analgesics. In Burger's Medicinal Chemistry and Drug Discovery, 5th ed.; Wolff, M. E., Ed.; John Wiley & Sons; New York, 1996; pp. 321-441). Fluorescent ligands can be used to label receptors in cell culture or tissue preparations and studied by fluorescence microscopy, confocal laser microscopy or flow cytometry. Strategically labelled ligands (e.g., with a fluorescent label) have been used as pharmacological tools to study receptor function and to aid in the identification of individual receptor types. In addition, they were utilized to assess the kinetics of receptor-ligand association and dissociation rates (Carraway et al., Biochemistry, 32: 12039-12045 (1993)), as well as the interactions between ligands, receptors, and G-proteins (Fay et al., Biochemistry, 30: 5066-5075 (1991); and Tota et al., Biochemistry, 33: 13079-13086 (1994)). Other receptor properties, such as the localization of the receptor-binding domain (Carraway et al., Biochemistry, 29: 8741-8747 (1990)) have also been examined using fluorescently labelled ligands.

Peptide ligands for opioid receptors were previously labelled with fluorescent functionalities, such as rhodamine (Hazzum et al., Biochem. Biophys. Res. Commun., 88: 841-846 (1979)), pyrene (Mihara et al., FEBS Lett., 193: 35-38 (1985)), dansyl (Berezowska et al., Peptides, 24: 1195-1200 (2003); and Berezowska et al., Acta Biochimica Polonica, 51: 107-113 (2004)), and fluorescein (Goldstein et al., Proc. Natl. Acad. Sci., U.S.A. 85: 7375-7379 (1988); and Kshirsagar et al., Neuroscience Letters, 249: 83-86 (1998)). These groups can be readily attached to either a free carboxylic acid or an amino group on the peptides in one of two ways: (i) to a side chain functional group of a non-critical residue, or (ii) by extending the peptide backbone in a manner which has minimal influence on binding at the ligand-binding domain (Kumar et al., J. Med. Chem., 43: 5050-5054 (2000)).

A non-peptide fluorescent probe, derived from the naltrindole template for the δ-opioid receptor, is a potent δ-opioid receptor antagonist in the mouse vas deferens (MVD) (smooth muscle) assay and binds to the δ-opioid receptor with relatively high affinity (Kᵢ=1 nM) and selectivity (Kshirsagar et al., Neuroscience Letters, 249: 83-86 (1998)). However, with the exception of the arylacetamide-derived fluorescent ligands (Chang et al., J. Med. Chem., 39: 1729-1735 (1996)), none of these compounds have been reported to be employed as molecular probes, nor was their selectivity for any of the major opioid receptor types (δ, μ, κ) demonstrated. Recently, Schiller et al. reported highly potent fluorescent analogues of the μ-opioid receptor peptide [Dmt¹]DALDA containing dansyl or anthranoyl fluorophores (Berezowska et al., Peptides, 24: 1195-1200 (2003); and Berezowska et al., Acta Biochimica Polonica, 51: 107-113 (2004)).

In view of the above, the present invention seeks to provide potent and/or selective fluorescently labeled opioid peptides as a pharmacological tool to study δ-opioid receptor structure and function. These and other advantages of the invention, as well as additional inventive features, will be apparent from the description of the invention provided herein.

### BRIEF SUMMARY OF THE INVENTION

The invention provides fluorescent compounds comprising Dmt-Tic, a group comprising one or more amino acid residues, a spacer, and a fluorescent molecule. The invention also provides compositions comprising such fluorescent compounds and at least one carrier.

The fluorescent compounds interact with δ- and μ-opioid receptors with high affinity and can be used to determine the number, structure, and/or activity of δ-opioid receptors in a tissue isolated from a subject. Thus, the invention also provides a method of identifying a δ-opioid or μ-opioid receptor in a mammal, which method comprises administering to the mammal at least one compound of formula: 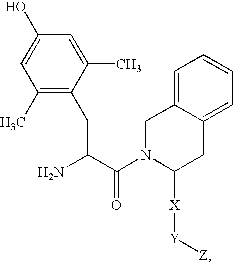
and detecting binding of the compound to the δ-opioid or μ-opioid receptor, wherein X is a group comprising one or more amino acid residues, Y is a spacer, and Z comprises a fluorescent molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a method of synthesis of a fluorescein bound tripeptide Dmt-Tic-Glu.

FIG. 2 depicts the inhibition of the electrically-evoked twitch by deltorphin C (DELT) of compound 3 of the invention at two concentrations on mouse vas deferens (MVD).

FIGS. 3A and 3B depict the confocal microscopic visualization of the fluorescence of compound 3 in NG108-15 cells. FIG. 3A shows the fluorescent photomicrograph of NG108-15 cells with fluorescence compound 3 and FIG. 3B shows cells preincubated with the δ-opioid receptor antagonist naltrindole.

### DETAILED DESCRIPTION OF THE INVENTION

The present invention provides compounds of formula: 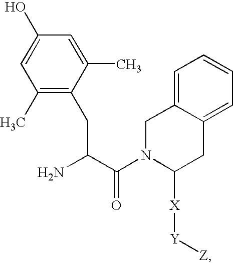
wherein X is a group comprising one or more amino acid residues, Y is a spacer, and Z comprises a fluorescent molecule.

The present inventive compounds can be synthesized by any suitable method. See, for example, Modern Techniques of Peptide and Amino Acid Analysis, John Wiley & Sons, 1981; Bodansky, Principles of Peptide Synthesis, Springer Verlag, 1984). Specific examples of the synthesis of the present inventive compounds are set forth in the Examples herein.

X comprises one or more amino acid residues, which comprise an amino group and a carbonyl, preferably in the form of an amide group. Preferably, either an acidic or amino functional group is at the terminal position that enables the spacer Y to be bound and still maintain a high δ-opioid receptor activity of the compound. The amino acid residue, which can be natural or synthetic, is preferably one of the twenty naturally occurring amino acids (e.g., methionine, threonine, cysteine, serine, alanine, valine, leucine, isoleucine, phenylalanine, tyrosine, histidine, tryptophane, aspartic acid, asparagine, glutamic acid, glutamine, lysine, arginine, glycine, and proline). More preferably, X comprises glutamic acid and/or aspartic acid. Preferably X comprises 1-6 amino acid residues, further preferably 1-3 amino acid residues, more preferably 1-2 amino acid residues, and even more preferably 1 amino acid residue. In some embodiments, X does not exist.

The spacer Y can be any suitable moiety, e.g. an organic moiety, that sufficiently binds the fluorescent molecule to the Dmt-Tic pharmacophore and reduces the influence of the fluorescent molecule on potential interference with opioid receptor affinity. For example, Y comprises an alkylenyl group of the formula —(CH₂)ₙ—, in which n is 0 to 10. Preferably, n is 1 to 8 (e.g., 1, 2, 3, 4, 5, 6, 7, or 8), and more preferably, n is 1 to 5 (e.g., 1, 2, 3, 4, or 5). Y can be substituted at the terminus and/or as a pendant group with one or more substituents, such as C₁₋₆ alkyl, C₃₋₈ cycloalkyl, aryl, heteroaryl, halo, hydroxy, amino, C₁₋₆ alkylamino, thiol, sulfido, carbonyl, and C═S. In addition, or alternatively, Y can comprise an unsubstituted or substituted ring (e.g., pyrazinonyl, piperazinyl, benzyl), in particular, a ring substituted with an aminoalkyl group. For examples of spacers comprising a ring compound (e.g., pyrazinonyl), see Okada et al., Chem. Pharm. Bull., 46: 1374-1382 (1998); Okada et al., Chem. Pharm. Bull., 46: 1374-1382 (1999); Okada et al., Chem. Pharm. Bull., 47: 1193-1195 (1999); Okada et al., Tetrahedron, 55: 14391-14406 (1999); Okada et al., Tetrahedron Lett., 43: 8137-8139 (2002); Okada et al., J. Med Chem., 46: 3509-3516 (2003); Jinsmaa et al., J. Pharmacol. Exp. Ther., 309: 1-7 (2004); Jinsmaa et al., J. Med. Chem., 47: 2599-2610 (2004), which have been incorporated by reference herein.

In embodiments of the invention, Y will be formed by a moiety added from the peptide side of the compound, such as the NH group of H-Dmt-Tic-Glu-NH, plus a moiety added from the fluorescent molecule side of the compound, such as the NH group from fluorescein. Thus, for example, if Z is fluorescein, Y can be —NH(CH₂)₅NHC(═S)NH—, which can be synthesized, in part from fluorescein isothiocyanate isomer I.

Z can be any moiety that generates UV-Vis radiation only when excited by a source of radiation having a wavelength different from the emitted wavelength. Z can be rhodamine, pyrene, dansyl, fluorescein, or anthranoyl. In a preferred embodiment, Z is fluorescein, including any of its isomers. Fluorescein is optimally excited at 490 nm and emits at 520 nm.

A preferred compound of the present invention has the formula 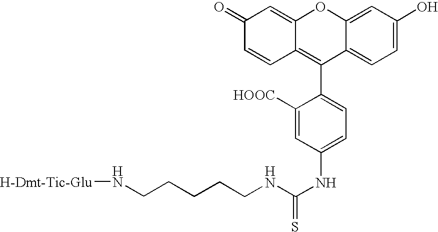
or an isomer thereof.

Whether an above-described compound functions as an agonist, a partial agonist, an antagonist, a partial antagonist, or a mixed agonist/antagonist is set forth, in part, in the Examples herein. Additionally, conventional techniques known to those of ordinary skill in the art can be used to make such determinations. Examples of such techniques include, but are not limited to, the mouse vas deferens in vitro assay of δ-receptors and the guinea pig ileum in vitro assay of μ-receptors as described in the Examples. Examples of in vivo studies include, but are not limited to, the tail flick test (Harris et al., J. Pharmacol. Meth., 20: 103-108 (1988); and Sing et al., P. A. Amber (v. 3.0. rev. A), Dept. Pharm. Chem., University of California, San Francisco, 1988).

The present invention further provides a composition comprising at least one of the above compounds. Desirably, the composition comprises at least one carrier, which is preferably a pharmaceutically acceptable carrier, diluent or vehicle. Also, desirably, the composition is formulated for human administration. Pharmaceutically acceptable carriers are well-known to those of ordinary skill in the art, as are suitable methods of administration. The choice of carrier will be determined, in part, by the particular method used to administer the composition. One of ordinary skill in the art will also appreciate that various routes of administering a composition are available, and, although more than one route can be used for administration, a particular route can provide a more immediate and more effective reaction than another route. Accordingly, there are a wide variety of suitable formulations of compositions that can be used in the present inventive methods.

A compound of the present invention can be made into a formulation suitable for parenteral administration, preferably intraperitoneal administration, or dural administration. Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be presented in unit dose or multi-dose sealed containers, such as ampules and vials, and can be stored in a freeze-dried (lyophilized) condition requiring only the addition of the sterile liquid carrier, for example, water, for injections, immediately prior to use. Extemporaneously injectable solutions and suspensions can be prepared from sterile powders, granules, and tablets, as described herein.

A formulation suitable for oral administration can consist of liquid solutions, such as an effective amount of the compound dissolved in diluents, such as water, saline, or fruit juice; capsules, sachets or tablets, each containing a predetermined amount of the active ingredient, as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

Similarly, a formulation suitable for oral administration can include lozenge forms, which can comprise the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, gels, and the like containing, in addition to the active ingredient, such carriers as are known in the art.

An aerosol formulation suitable for administration via inhalation also can be made. The aerosol formulation can be placed into a pressurized acceptable propellant, such as dichlorodifluoromethane, propane, nitrogen, and the like.

A formulation suitable for topical application can be in the form of creams, ointments, or lotions.

A formulation for rectal administration can be presented as a suppository with a suitable base comprising, for exanple, cocoa butter or a salicylate. A formulation suitable for vaginal administration can be presented as a pessary, tampon, cream, gel, paste, foam, or spray formula containing, in addition to the active ingredient, such carriers as are known in the art to be appropriate.

Any of the above compositions can further comprise one or more other active agents. Alternatively, any of the above compositions can be administered, by the same or different route, in combination with another composition comprising one or more other active agents, either simultaneously or sequentially in either order sufficiently close in time to realize the benefit of such co-administration. Additional active agents include, for example, pain relievers, including non-steroidal anti-inflammatory drugs (NSAIDs) (e.g., acetaminophen, aspirin, methyl salicylate, diflunisal, indomethacin, sulindac, diclofenac, ibuprofen, ketoprofen, naproxen, ketorolac, meloxicam, piroxicam, celecoxib, valdecoxib, parecoxib, etoricoxib), and corticosteroids (e.g., cortisone, hydrocortisone, prednisone, prednisolone, triamcinolone, methylprednisolone, dexamethasone, betamethasone).

The fluorescent Dmt-Tic compounds of the present invention can be used to determine the number, structure, and/or activity of δ-opioid receptors in a tissue isolated from a subject (e.g., a human). Information gleaned from such investigations can be used to predict the efficacy of a therapeutic candidate in alleviating pain, for example, or to predict an individual's response to a therapeutic candidate. Once identified using a method of the invention, a therapeutic candidate can be used in the treatment of chronic or acute pain, alcoholism, genetically-derived symptoms such as autism, neurological diseases, and neuropeptide or neurotransmitter imbalances.

In particular, the inventive compounds can be used as probes to study the in vitro localization and/or distribution of δ-opioid receptors in tissues, the internalization of opioid peptides during signal transduction, and/or δ-opioid trafficking in live cells. Such properties can be assessed using any suitable method in the art, such as, for example, fluorescence microscopy, confocal laser microscopy or flow cytometry (see, e.g., Arttamangkul et al., Mol. Pharmacol., 58: 1570-1580 (2000), and U.S. Pat. No. 4,661,913). In a preferred embodiment of the invention, binding of the inventive compounds to the δ-opioid receptor is visualized in real time using confocal laser microscopy.

Thus, present invention further provides a method of identifying a δ-opioid receptor in a mammal, which method comprises administering to the mammal at least one compound of formula: 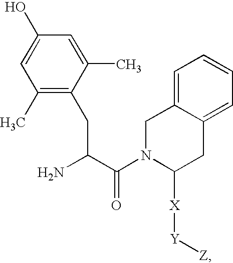
and detecting binding of the compound to the δ-opioid receptor, wherein X is a group comprising one or more amino acid residues, Y is a spacer, and Z comprises a fluorescent molecule.

The present invention also provides a method of identifying a μ-opioid receptor in a mammal, which method comprises administering to the mammal at least one compound of formula: 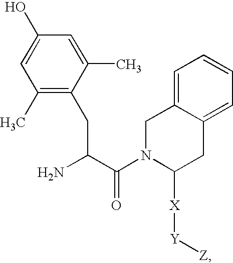
and detecting binding of the compound to the μ-opioid receptor, wherein X is a group comprising one or more amino acid residues, Y is a spacer, and Z comprises a fluorescent molecule.

In embodiments of the invention is provided a method of identifying a δ-opioid or μ-opioid receptor in a sample, which method comprises contacting the sample with at least one compound of formula: 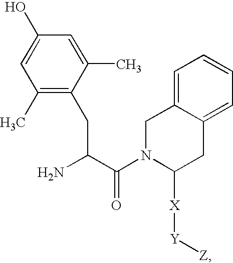
wherein X is a group comprising one or more amino acid residues, Y is a spacer, and Z comprises a fluorescent molecule, and detecting binding of the compound to the δ-opioid or μ-opioid receptor. The sample can be any suitable sample in which a δ-opioid or μ-opioid receptor could be found. The sample can be, for example, a tissue, blood, or serum. The tissue can be isolated from any suitable organ in a mammal (e.g., human), including the heart, brain, reproductive organs (e.g., uterus), or digestive organs (e.g., stomach, intestines).

Detecting binding of the compound to a δ-opioid receptor or a μ-opioid receptor can be performed using any suitable method to detect ligand-receptor interactions. Such methods are well known to those skilled in the art, and include, for example, flow cytometry, competitive inhibition assay, immunofluorescence microscopy, immunoelectron microscopy, and confocal laser microscopy. Such methods are described in, for example, U.S. Pat. No. 4,661,913, Arttamangkul et al., supra, and Cechetto et al., Exp Cell Res., 260: 30-39 (2000). One of ordinary skill in the art will appreciate that binding of the inventive compound to a δ-opioid receptor or a μ-opioid receptor can antagonize or agonize the δ- or μ-opioid signaling pathway, respectively.

The term “antagonist,” as used herein, refers to a compound that bears sufficient structural similarity to an endogenous δ-opioid or μ-opioid ligand to compete with the endogenous ligand and inhibit δ- or μ-opioid signaling. In contrast, the term “agonist,” as used herein, refers to a compound that bears sufficient structural similarity to an endogenous δ-opioid or μ-opioid ligand to compete with the endogenous ligand and activate or enhance δ- or μ-opioid signaling.

The specificity and affinity of the inventive compounds for δ-opioid receptors can be determined using any suitable method, such as a non-radiolabelled competitive binding assay (see, e.g., Balboni et al., J. Med. Chem., 45: 5556-5563 (2002), Lazarus et al., J. Med Chem., 34: 1350-1359 (1991), Salvadori et al., J. Med. Chem., 42: 5010-5019 (1999), and Balboni et al., Bioorg. Med. Chem., 11: 5435-5441 (2003)).

In embodiments of the invention, it is contemplated that analogues of the Dmt-Tic pharmacophore minus the fluorescent moiety can be useful in medicinal applications. Such applications include the treatment of chronic or acute pain, alcoholism, genetically-derived symptoms such as autism, neurological diseases, and neuropeptide or neurotransmitter imbalances.

The dose administered to a mammal, particularly a human, in the context of the present invention should be sufficient to affect a response, including a therapeutic response, in the individual over a reasonable time frame. The dose will be determined by the potency of the particular compound employed for treatment, the severity of any condition to be treated, as well as the body weight and age of the individual. The size of the dose also will be determined by the existence of any adverse side effects that may accompany the use of the particular compound employed. It is always desirable, whenever possible, to keep adverse side effects to a minimum.

The dosage can be in unit dosage form, such as a tablet or capsule. The term “unit dosage form” as used herein refers to physically discrete units suitable as unitary dosages for human and animal subjects, each unit containing a predetermined quantity of a compound, alone or in combination with other active agents, calculated in an amount sufficient to produce the desired effect in association with a pharmaceutically acceptable diluent, carrier, or vehicle. The specifications for the unit dosage forms of the present invention depend on the particular embodiment employed and the effect to be achieved, as well as the pharmacodynamics associated with each compound in the host. The dose administered should be an effective amount, i.e., an amount effective to antagonize or agonize a δ-opioid receptor or a μ-opioid receptor as desired.

Since the “effective amount” is used as the preferred endpoint for dosing, the actual dose and schedule can vary, depending on interindividual differences in pharmacokinetics, drug distribution, and metabolism. The “effective amount” can be defined, for example, as the blood or tissue level desired in the patient that corresponds to a concentration of one or more compounds according to the invention. The “effective amount” for a given compound of the present invention also can vary when the composition of the present invention comprises another active agent or is used in combination with another composition comprising another active agent.

One of ordinary skill in the art can easily determine the appropriate dose, schedule, and method of administration for the exact formulation of the composition being used, in order to achieve the desired “effective amount” in the individual patient. One skilled in the art also can readily determine and use an appropriate indicator of the “effective amount” of the compound of the present invention by pharmacological end-point analysis.

Further, with respect to determining the effective amount in a patient, suitable animal models are available and have been widely implemented for evaluating the in vivo efficacy of such compounds. These models include the tail flick test (see, e.g., U.S. Pat. No. 5,780,589). In vitro models are also available, examples of which are set forth in the Examples herein.

Generally, an amount of a present inventive compound up to about 50 mg/kg body weight, preferably from about 10 mg/kg body weight to about 50 mg/kg body weight is preferred, especially from about 10 mg/kg body weight to about 20 mg/kg body weight. In certain applications, multiple daily doses are preferred. Moreover, the number of doses will vary depending on the means of delivery and the particular compound administered.

### ABBREVIATIONS

DAMGO [D-Ala²,N-Me-Phe⁴,Gly-ol⁵] enkephalin
Bid 1H-benzimidazol-2-yl
Boc tert-butyloxycarbonyl
DELT or deltorphin C [D-Ala²]deltorphin I (Tyr-D-Ala-Phe-Asp-Val-Val-Gly-NH₂)
Dmt 2′,6′-dimethyl-L-tyrosine
DPDPE cyclic[D-Pen^{2,5}]enkephalin
GPI guinea-pig ileum
HOBt 1-hydroxybenzotriazole
HPLC high performance liquid chromatography
MALDI-TOF matrix assisted laser desorption ionization time of flight
MVD mouse vas deferens
pA₂ negative log of the molar concentration required to double the agonist concentration to achieve the original response
TFA trifluoroacetic acid
Tic 1,2,3,4-tetrahydroisoquinoline-3-carboxylic acid
TIP(P) H-Tyr-Tic-Phe-(Phe)-OH
TLC thin-layer chromatography
WSC 1-ethyl-3-(3′-dimethylaminopropyl)-carbodiimide-HCl

### EXAMPLES

The following examples further illustrate the invention but, of course, should not be construed as in any way limiting its scope.

Crude fluorescein-tripeptide was purified by preparative reversed-phase high-performance liquid chromatography (HPLC) using a Waters Delta Prep 4000 system with Waters PrepLC 40 mm Assembly column C₁₈ (30×4 cm, 15 μm particle size column). The column was perfused at a flow rate of 40 mL/min with mobile phase solvent A (10% acetonitrile in 0.1% TFA, v/v), and a linear gradient from 0 to 50% of solvent B (60%, acetonitrile in 0.1% TFA, v/v) in 25 min was adopted for the elution of the products. Analytical HPLC analyses were performed using a Beckman System Gold and a Beckman ultrasphere ODS colunm (250×4.6 mm, 5 μm particle size). Analytical determinations and capacity factor (K′) of the products were determined using HPLC conditions in the above solvent systems (solvents A and B) programmed at flow rate of 1 mL/min using the following linear gradient: from 0 to 50% B in 25 min. All analogues showed less than 1% impurities when monitored at 220 and 254 nm. TLC was performed on precoated plates of silica gel F254 (Merck, Darmstadt, Germany) using the following solvent systems: (A) 1-butanol/AcOH/H₂O (3:1:1, v/v/v); and (B) CH₂Cl₂/toluene/methanol (17:1:2, v/v/v). Ninhydrin (1%, Merck), fluorescamine (Hoffman-La Roche) and chlorine reagents were used as sprays. Open column chromatography (2×70 cm, 0.7-1 g material) was run on silica gel 60 (70-230 mesh, Merck) using the same eluent systems. Melting points were determined on a Kofler apparatus and are uncorrected. Optical rotations were determined at 10 mg/mL in methanol with a Perkin-Elmer 241 polarimeter with a 10 cm water-jacketed cell. All ¹H-NMR spectra were recorded on a Bruker 200 MHz spectrometer. MALDI-TOF analyses (matrix assisted laser desorption ionization time-of-flight mass spectrometry) of peptides were conducted using a Hewlett Packard G 2025 A LD-TOF system. The samples were analyzed in the linear mode with 28 kV accelerating voltage, mixing them with a saturated solution of α-cyano-4-hydroxycinnamic acid matrix.

### Example 1

This example illustrates the peptide synthesis of Boc-Glu(OBzl)—NH(CH₂)₅—NH-Z.

To a solution of Boc-Glu(OBzl)—OH (0.30 g, 0.90 mmol) and N-Z-1,5-pentanediamine hydrochloride (0.24 g, 0.90 mmol) in DMF (10 mL) at 0° C. were added NMM (0.10 mL, 0.90 mmol), HOBt (0.15 g, 0.99 mmol) and WSC (0.19 g, 0.99 mmol). Z is the protecting group benzyloxycarbonyl. The reaction mixture was stirred for 3 h at 0° C. and for 24 h at room temperature. After DMF was evaporated, the residue was solubilized in EtOAc and washed with citric acid (10%), NaHCO₃ (5%), and brine. The organic phase was dried and evaporated to dryness. The residue was crystallized from Et₂O/Pe (1:9, v/v): yield 0.47 g (94%); R_{f} (B) 0.94; HPLC K′=9.15; mp 141-143° C.; [α]²⁰_{D}+20.4; MH⁺ 556; ¹H NMR (DMSO) δ 1.29-1.55 (m, 15 H), 2.18-2.25 (m, 4H), 2.96-3.20 (m, 4H), 4.53-5.34 (m, 5H), 7.11-7.29 (m, 10H).

### Example 2

This example illustrates the peptide synthesis of TFA.H-Glu(OBzl)—NH(CH₂)₅—NH-Z.

Boc-Glu(OBzl)—NH(CH₂)₅—NH-Z (0.47 g, 0.85 mmol) was treated with TFA (2 mL) for 30 min. at room temperature. Et₂O/Pe (1:5, v/v) were added to the solution until the product precipitated: yield 0.46 g (94%); R_{f} (A) 0.77; HPLC K′=6.89; mp 153-155° C.; [α]²⁰_{D}+23.9; MH⁺ 456.

### Example 3

This example illustrates the peptide synthesis of Boc-Tic-Glu(OBzl)—NH(CH₂)₅—NH-Z.

To a solution of Boc-Tic-OH (0.22 g, 0.80 mmol) and TFA.H-Glu(OBzl)—NH(CH₂)₅—NH-Z (0.46 g, 0.80 mmol) in DMF (10 mL) at 0° C. were added NMM (0.09 mL, 0.80 mmol), HOBt (0.13 g, 0.88 mmol) and WSC (0.17 g, 0.88 mmol). The reaction mixture was stirred for 3 h at 0° C. and for 24 h at room temperature. After DMF was evaporated, the residue was treated as reported above for Boc-Glu(OBzl)—NH(CH₂)₅—NH-Z: yield 0.51 g (89%); R_{f} (B) 0.95; HPLC K′=9.26; mp 143-145° C.; [α]²⁰_{D}+16.7; MH⁺ 615; ¹H NMR (DMSO) δ 1.29-1.55 (m, 15 H), 2.18-2.25 (m, 4H), 2.96-3.20 (m, 6H), 4.22-5.34 (m, 8H), 6.96-7.19 (m, 14H).

### Example 4

This example illustrates the peptide synthesis of TFA.H-Tic-Glu(OBzl)—NH(CH₂)₅—NH-Z.

Boc-Tic-Glu(OBzl)—NH(CH₂)₅—NH-Z (0.51 g, 0.71 mmol) was treated with TFA (2 mL) for 30 min. at room temperature. Et₂O/Pe (1:5, v/v) were added to the solution until the product precipitated: yield 0.49 g (94%); R_{f} (A) 0.79; HPLC K′=6.85; mp 156-158° C.; [α]²⁰_{D}+18.1; MH⁺ 615.

### Example 5

This example illustrates the peptide synthesis of Boc-Dmt-Tic-Glu(OBzl)—NH(CH₂)₅—NH-Z.

To a solution of Boc-Dmt-OH (0.21 g, 0.67 mmol) and TFA.H-Tic-Glu(OBzl)—NH(CH₂)₅—NH-Z (0.49 g, 0.67 mmol) in DMF (10 mL) at 0° C. were added NMM (0.07 mL, 0.67 mmol), HOBt (0.11 g, 0.74 mmol) and WSC (0.14 g, 0.74 mmol). The reaction mixture was stirred for 3 h at 0° C. and for 24 h at room temperature. After DMF was evaporated, the residue was treated as reported above for Boc-Glu(OBzl)—NH(CH₂)₅—NH-Z: yield 0.40 g (88%); R_{f} (B) 0.87; HPLC K′=8.94; mp 140-142° C.; [α]²⁰_{D}+17.1; MH⁺ 905; ¹H NMR (DMSO) 6 1.29-1.55 (m, 15 H), 2.06-2.35 (m, 10H), 2.96-3.20 (m, 8H), 4.40-5.34 (m, 9H), 6.29 (s, 2H), 6.96-7.19 (m, 14H).

### Example 6

This example illustrates the peptide synthesis of Boc-Dmt-Tic-Glu—NH(CH₂)₅—NH₂.

To a solution of Boc-Dmt-Tic-Glu(OBzl)—NH(CH₂)₅—NH-Z (0.4 g, 0.44 mmol) in methanol (30 mL) was added C/Pd (10%, 0.07 g) and H₂ was bubbled for 1 h at room temperature. After filtration, the solution was evaporated to dryness. The residue was crystallized from Et₂O/Pe (1:9, v/v): yield 0.27 g (90%); R_{f} (A) 0.58; HPLC K′=3.87; mp 161-163° C.; [α]²⁰_{D}+19.4; MH⁺ 682.

### Example 7

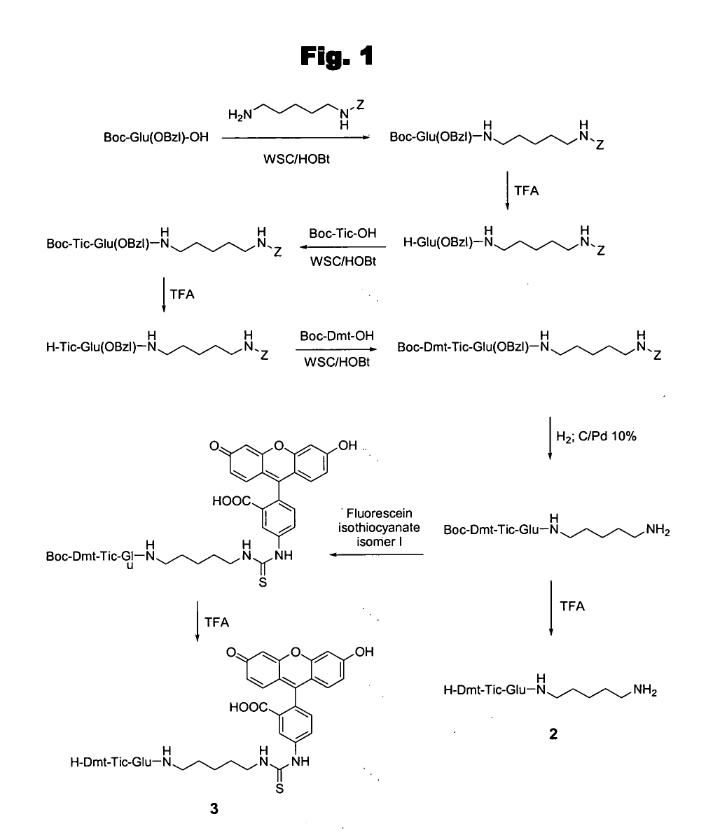

This example illustrates the peptide synthesis of 2TFA.H-Dmt-Tic-Glu—NH(CH₂)₅—NH₂ (2). See FIG. 1.

Boc-Dmt-Tic-Glu—NH(CH₂)₅—NH₂ (0.05 g, 0.07 mmol) was treated with TFA (1 mL) for 30 min. at room temperature. Et₂O/Pe (1:5, v/v) were added to the solution until the product precipitated: yield 0.06 g (95%); R_{f} (A) 0.59; HPLC K′=4.21; mp 163-165° C.; [α]²⁰_{D}+19.4; MH⁺ 582; ¹H NMR (DMSO) 6 1.29-1.55 (m, 6 H), 2.05-3.20 (m, 18H), 3.95-4.92 (m, 5H), 6.29 (s, 2H), 6.96-7.02 (m, 4H).

### Example 8

This example illustrates the peptide synthesis of 5-(3-{5-[2-({2-[2-tert-butoxycarbonylamino-3-(4-hydroxy-2,6-dimethyl-phenyl)-propionyl]-1,2,3,4-tetrahydroisoquinoline-3-carbonyl}-amino)-4-carboxy-butyrylamino]-pentyl}-thioureido)-2-(6-hydroxy-3-oxo-3H-xanten-9-yl)-benzoic acid [Boc-Dmt-Tic-Glu-NH(CH₂)₅—NH—(C═S)—NH-fluorescein].

With stirring at 25° C. under argon, fluorescein isothiocyanate isomer 1 (0.06 g, 0.15 mmol) was added to a mixture of Boc-Dmt-Tic-Glu-NH(CH₂)₅—NH₂ (0.1 g, 0.15 mmol) and triethylamine (2.5 mL) in freshly distilled THF (10 mL) and absolute ethanol (15 mL). The reaction mixture was stirred in the dark at room temperature for 24 h. After solvent evaporation, the residue was purified by preparative HPLC: yield 0.07 g (49%); R_{f} (B) 0.74; HPLC K′=8.03; mp 157-159° C.; [α]²⁰_{D}+8.2; MH⁺ 1070; ¹H NMR (DMSO) δ 1.29-1.55 (m, 15 H), 2.06-2.35 (m, 10H), 3.05-3.45 (m, 8H), 4.40-4.92 (m, 5H), 6.11-7.26 (m, 15H) (Chang et al., J. Med. Chem., 39: 1729-1735 (1996)).

### Example 9

This example illustrates the peptide synthesis of 5-(3-{5-[2-({2-[2-amino-3-(4-hydroxy-2,6-dimethyl-phenyl)-propionyl]-1,2,3,4-tetrahydro-isoquinoline-3-carbonyl}-amino)-4-carboxy-butyrylamino]-pentyl}-thioureido)-2-(6-hydroxy-3-oxo-3H-xanten-9-yl)-benzoic acid. [TFA.H-Dmt-Tic-Glu-NH(CH₂)₅—NH—(C═S)—NH-fluorescein] (3). See FIG. 1.

Boc-Dmt-Tic-Glu-NH(CH₂)₅—NHCSNH-fluorescein (0.07 g, 0.07 mmol) was treated with 66% TFA (1 mL) for 30 min. at room temperature. Et₂O/Pe (1:5, v/v) were added to the solution until the product precipitated: yield 0.067 g (94%); R_{f} (A) 0.71; HPLC K′=5.47; mp 169-171° C.; [α]²⁰_{D}+9.7; MH⁺ 971; ¹H NMR (DMSO) δ 1.29-1.55 (m, 6H), 2.05-2.35 (m, 10H), 3.05-3.95 (m, 9H), 4.46-4.92 (m, 4H), 6.11-7.28 (m, 15H) (Goldstein et al., Proc. Natl. Acad. Sci. USA, 85: 7375-7379 (1998)).

### Example 10

This example illustrates competitive receptor binding assays.

These assays were conducted as described in considerable detail elsewhere using rat brain synaptosomes (P₂ fraction) (Balboni et al., J. Med Chem., 45: 5556-5563 (2002); Lazarus et al., J. Med Chem., 34: 1350-1359 (1991); Salvadori et al., J. Med Chem., 42: 5010-5019 (1999); and Balboni et al., Bioorg. Med. Chem., 11: 5435-5441 (2003)). Membrane preparations were preincubated to eliminate endogenous opioid peptides and stored at −80° C. in buffered 20% glycerol (Lazarus et al., J. Med. Chem., 34: 1350-1359 (1991); and Lazarus et al., J. Biol. Chem., 264: 3047-3050 (1989)). Each analogue was analyzed in duplicate using 5 to 8 dosages of peptide and independent repetitions with different synaptosomal preparations (n values are listed in Table 1 in parenthesis and the results are listed as the mean±SE). Unlabeled peptide (2 μM) was used to determine non-specific binding in the presence of either 5.53 nM [³H]DPDPE (34.0 Ci/mmol, PerkinElmer, Boston, Mass.; K_{D}=4.5 nM) for δ-opioid receptors, and for μ-opioid receptors, 3.5 nM [³H]DAMGO (50.0 Ci/mmol, Amersham Biosciences, Buckinghamshire, UK; K_{D}=1.5 nM). Glass fiber filters (Whatman GFC) were soaked in 0.1% polyethyleneimine in order to enhance the signal:noise ratio of the bound radiolabeled-synaptosome complex, and the filters washed thrice in ice cold buffered BSA (Lazarus et al., J. Med Chem., 34: 1350-1359 (1991)). The affinity constants (Kᵢ) were calculated according to Cheng et al., Biochem. Pharmacol., 22: 3099-3108 (1973).

TABLE 1

| | | | | | Functional bioactivity | | |
|---|---|---|---|---|---|---|---|
| | | Receptor affinity^{a} (nm) | | | MVD | | GPI |
| | | | | | (IC₅₀)^{c} | MVD | (IC₅₀)^{c} |
| No. | Compound | Kᵢ^{δ} | Kᵢ^{u} | μ/δ | (nm) | pA₂^{b} | (μM) |
| 1 | H-Dmt-Tic-Glu-NH₂ | 0.06 ± 0.008 | 1360 ± 268 | 22600^{d} | 2.58 ± 0.8^{d} | | >1 |
| | | (4)^{d} | (4)^{d} | | | | |
| 2 | H-Dmt-Tic-Glu-NH-(CH₂)₅—NH₂ | 0.22 ± 0.04 | 380 ± 65 | 1700 | | 8.8 | >1 |
| | | (4) | (4) | | | | |
| 3 | 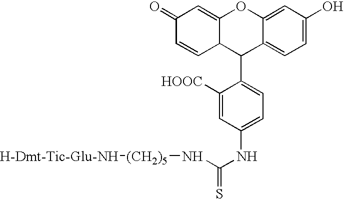 | 0.035 ± 0.01 (4) | 152 ± 44 (4) | 4370 | | Irreversible antagonist | >1 |
| ^{a}Ki values (nM) were determined according to Cheng et al., Biochem. Pharmacol., 22, 3099-3108 (1973). The mean ± SE with n repetitions in parentheses is based on independent duplicate binding assays with five to eight | | | | | | | |
| # peptide doses using several different synaptosomal preparations. | | | | | | | |
| ^{b}pA₂ is the negative logarithm to base 10 of the molar concentration of an antagonist that is necessary to double the concentration of agonist needed to elicit the original submaximal response; the antagonist properties of these | | | | | | | |
| # compounds were tested using deltorphin C (δ-opioid receptor agonist) or dermorphin (μ-opioid receptor agonist). | | | | | | | |
| ^{c}Agonist activity was expressed as IC₅₀ obtained from dose-response curves. These values represent the mean ± SE for at least five fresh tissue samples. Deltorphin C and dermorphin were the internal standards for MVD (δ-opioid | | | | | | | |
| # receptor bioactivity) and GPI (μ-opioid receptor bioactivity) tissue preparations, respectively. | | | | | | | |

^{d}Data taken from Balboni et at., J. Med Chem., 47: 4066-4071 (2004).

In the receptor binding assays, the fluorescent probe, H-Dmt-Tic-Glu-NH—(CH₂)₅—NH—(C═S)—NH-fluorescein (3), displayed subnanomolar δ-opioid receptor binding affinity, which lies within the same order of magnitude as the reference compound H-Dmt-Tic-Glu-NH₂ (1) while the tripeptide (2), containing only the spacer at the C-terminus, exhibited only a 3.7-fold decrease in affinity for δ-opioid receptors. The μ-opioid receptor affinity increased 3.6- and ca. 9-fold for 2 and 3, respectively, compared to the reference tripeptide (1). As a consequence the δ-opioid receptor selectivity of the fluorescent compound 3 fell 5-fold, from 22,600 to 4,370 and that of the tripeptide 2 decreased 13-fold compared to reference 1.

While a direct comparison between the δ-opioid receptor selectivity of a fluorescent probe of the present invention and that of other fluorescent opioid molecules found in the literature may not be wholly compatible due to inherent differences in assay methods, it is nonetheless instructive to compare them when inconsistencies exceed orders of magnitude: compound 3, for example, was 115- and 857-fold more selective than fluorescent naltrindole derivatives (Kshirsagar et al., Neuroscience Letters, 249: 83-86 (1998); and Korlipara et al., Eur. J. Med. Chem., 32: 171-174 (1997)). Similarly, the labelling of the δ-opioid receptor agonist [D-Ala²]deltorphin I with Alexa 488 and BODIPY TR caused a precipitous loss of δ-selectivity from 9,000 to >128 and 16, respectively. Moreover, TIPP, another δ-opioid selective antagonist labelled with Alexa 488 exhibited a marked change in selectivity from >20,000 to 84 (Arttamangkul et al., Mol. Pharmacol., 58: 1570-1580 (2000)).

### Example 11

This example illustrates the functional bioactivity in isolated organ preparations.

Preparations of myenteric plexus-longitudinal muscle obtained from male guinea-pig ileum (GPI, enriched in μ-opioid receptors) and preparations of mouse vas deferens (MVD, containing δ-opioid receptors) were used for field stimulation with bipolar rectangular pulses of supramaximal voltage (Melchiorri et al., Eur. J. Pharm., 195: 201-207 (1991)). Agonists were evaluated for their ability to inhibit the electrically-evoked twitch. The biological potency of the compounds was compared with that of the μ-opioid receptor agonist dermorphin in GPI and with that of the δ-opioid receptor agonist deltorphin C in MVD. The results are expressed as the IC₅₀ values obtained from dose-response curves (Prism™, GraphPad). To evaluate antagonistic properties, compounds 2 and 3 were added to the bath and allowed to interact with tissue receptor sites 5 min before adding deltorphin C. The IC₅₀ values (nM) represent the mean of not less than six fresh tissue samples±SE. Competitive antagonist activities were evaluated for their ability to shift the deltorphin C (MVD) and dermorphin (GPI) log-concentration-response curve to the right; pA₂ values were determined using the Schild Plot (Tallarida, R. J.; Murray, R. B. Manual of Pharmacological Calculation; 2^{nd} ed.; Springer-Verlag: New York, 1986). IC₅₀ and pA₂ values (nM) are mean ±SE of at least six experiments conducted with fresh tissues. See Table 1 above.

In the in vitro functional bioactivity profiles of compounds 1-3, there was negligible activity in the GPI preparations (IC₅₀>1 μM). In the MVD assay, tripeptide 1 was a partial δ-opioid agonist (Balboni et al., J. Med. Chem., 47: 4066-4071 (2004)), and C-terminal amidation with a spacer, as demonstrated with compound 2, transformed the intrinsic δ-opioid agonist activity into δ-opioid antagonist activity: it behaved as a competitive antagonist producing parallel displacing of [D-Ala²]deltorphin I dose-response curves without alteration of the maximal response, from which equiactive dose ratios can be calculated and used in the Schild equation (pA₂=8.8).

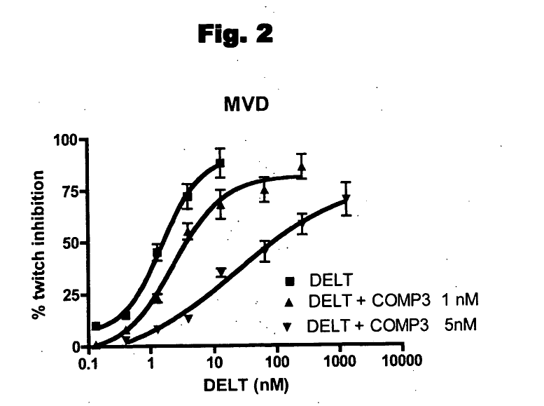

The fluorescent derivative 3 in the MVD had non-equilibrium antagonist activity (see FIG. 2). The log dose-response curves of [D-Ala²]deltorphin I in the presence of increasing concentrations of 3 reflected a reduction in the apparent efficacy and Hill slope ([D-Ala²]deltorphin I=1.4; +1 nM 3=1.1; +5 nM 3=0.5). The compound bound tightly and dissociated very slowly from the tissue preparation: antagonism could not be reversed by washing the tissue with a drug-free solution for a time exceeding three hours; moreover the longer the compound was in contact with the tissue, the greater was the magnitude of the observed antagonism.

### Example 12

This example describes the fluorescence emission spectra of compound 3.

Fluorescence emission spectra were recorded on a Jobin Yvon-Spex FluoroMax-2 spectrofluorometer with 1 nm spectral resolution for excitation and emission. A peptide solution at a concentration of 2×10⁻⁵M in Tris-HCl buffer, pH 6.6, was used. The excitation wavelength was 350 nm. Fluorescence quantum yield (φ) was determined relative to quinine sulfate (Fluka) in 1 N H₂SO₄ (φ=0.546) as a reference (Meech et al., Journal of Photochemistry, 23: 193 (1983)). The quantum yield was calculated according to the following equation:

$${φ}_{S} = {φ}_{R} \frac{{E}_{S} {A}_{R}}{{E}_{R} {A}_{S}} {( \frac{{n}_{S}}{{n}_{R}} )}^{2}$$

where the subscripts S and R refer to the sample nₛ and reference compound, respectively; E is the integrated area under the corrected emission spectrum; A is the absorbance of the solution at the excitation wavelength. Absorbance values were kept below 0.02 to minimise inner filter and self-quenching effects. Since both the sample and the reference were in aqueous solution, the correction for the refractive index (n_{S}/n_{R})² was considered to be of no significant relevance.

The fluorescence emission spectra of H-Dmt-Tic-Glu-NH(CH₂)₅—NH—(C═S)—NH-fluorescein and the reference amino acid derivative Ac-Glu-NH(CH₂)₅—NH—(C═S)—NH-fluorescein both show a maximum at 515 nM in Tris/HCl buffer (pH 6.6). This indicated that the fluorescein label of the tripeptide was located in a completely aqueous environment and did not engage in any significant intramolecular interactions. This was also verified by the similar fluorescence quantum yields calculated for the fluorescein-tripeptide (φ=0.227) and for the reference fluorescein-amino acid derivative Ac-Glu-NH(CH₂)₅—NH—(C═S)—NH-fluorescein.

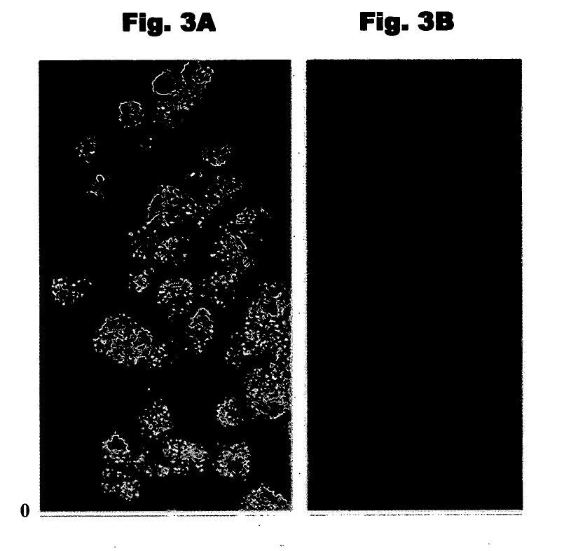

Visualization of δ-opioid receptor sites with the inventive fluorescent probe was obtained by incubating (15 min at 35° C.) the fluorescent tripeptide 3 (0.2 μM) with the NG108-15 (mouse N18 neuroblastoma x rat C6 glioma) cell line, which expresses mouse δ-opioid receptors. The left panel of FIG. 3 reveals the fluorescent photomicrograph obtained from the confocal scanning laser microscope, while in the right panel the fluorescent photomicrograph is that of cells preincubated with the δ-opioid receptor antagonist naltrindole (0.2 μM) before addition of the fluorescent probe (0.2 nM); naltrindole essentially eliminated the fluorescence bound to δ-opioid receptors. Localization and visualization of opioid receptor binding sites were obtained by incubating the fluorescent probe with the NG108-15 cells as shown in the left half of the fluorescent photomicrograph obtained from the confocal scanning laser microscope (FIG. 3). Preincubation of the cells with δ-opioid receptor antagonist NTI for 5 min prior to adding the fluorescent probe 3, produced considerable blockage of the fluorescence as evidenced in the right half of FIG. 3. Similar results were obtained when the competition experiments were performed with other δ-opioid receptor antagonists, such as N,N′(CH₃)₂-Dmt-Tic-OH and TIPP. However, the residual fluorescence (ca. 10%) suggests a minor non-specific binding of the probe to NG108-15 cell membranes, or a residual staining of a fraction of δ-opioid receptors due to the essentially irreversible kinetics of the fluorescent probe. The high lipophilicity of fluorescein may contribute to this non-specific binding to NG108-15 lipid membranes and to the distinct pharmacological behaviour of the fluorescent probe in the MVD assay.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula: 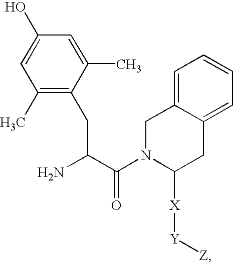
wherein X is a group comprising one or more amino acid residues, Y is a spacer, and Z comprises a fluorescent molecule.

2. The compound of claim 1, wherein X comprises at least one of the twenty naturally occurring amino acids.

3. The compound of claim 1, wherein Y comprises an alkyl group of the formula —(CH₂)ₙ—, wherein n is 0 to 10, and wherein Y is optionally substituted at the terminus and/or as a pendant group with one or more substituents selected from the group consisting of C₁₋₆ alkyl, C₁₋₈cycloalkyl, aryl, heteroaryl, halo, hydroxy, amino, alkylamino, mercapto, sulfido, carbonyl, and C═S.

4. The compound of claim 1, wherein Y comprises pyrazinonyl, piperazinyl, or benzyl.

5. The compound of claim 1, wherein Z comprises rhodaminyl, pyrenyl, dansyl, fluoresceinyl, or anthranoyl.

6. The compound of claim 1, wherein the compound has the formula

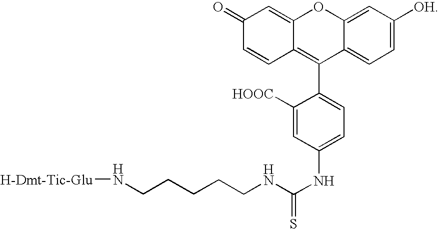

7. A composition comprising at least one compound of claim 1 and at least one carrier.

8. A method of identifying a δ-opioid receptor in a mammal, which method comprises administering to the mammal at least one compound of claim 1 and detecting binding of the compound to the δ-opioid receptor.

9. A method of identifying a μ-opioid receptor in a mammal, which method comprises administering to the mammal at least one compound of claim 1 and detecting binding of the compound to the μ-opioid receptor.

10. A method of identifying a δ-opioid receptor in a sample, which method comprises contacting the sample with at least one compound of claim 1 and detecting binding of the compound to the δ-opioid receptor.

11. A method of identifying a μ-opioid receptor in a sample, which method comprises contacting the sample with at least one compound of claim 1 and detecting binding of the compound to the μ-opioid receptor.

12. The method of claim 8, wherein the receptor is detected using flow cytometry, competitive inhibition assay, immunofluorescence microscopy, immunoelectron microscopy, or confocal laser microscopy.

13. The method of claim 10, wherein the sample is a tissue.

14. The method of claim 11, wherein the sample is a tissue.

15. A composition comprising at least one compound of claim 6 and at least one carrier.

16. A method of identifying a δ-opioid or μ-opioid receptor in a mammal, which method comprises administering to the mammal at least one compound of claim 6 and detecting binding of the compound to the δ-opioid or μ-opioid receptor.

17. A method of identifying a δ-opioid or μ-opioid receptor in a sample, which method comprises contacting the sample with at least one compound of claim 6 and detecting binding of the compound to the δ-opioid or μ-opioid receptor.

18. The method of claim 17, wherein the sample is a tissue.

19. The method of claim 2, wherein X comprises glutamic acid and/or aspartic acid.
